(19) United States
(12) Patent Application Publication
(10) Pub. No.: US 2009/0291212 A1
(43) Pub. Date: Nov. 26, 2009

(54) VOLATILE METAL COMPLEXES OF PERFLUORO-tert-BUTANOL

(75) Inventors: William D. Buchanan, LaFayette, NY (US); Karin Ruhlandt-Senge, Homer, NY (US)

Correspondence Address: Hiscock & Barclay, LLP, One Park Place, 300 South State Street, Syracuse, NY, 13202-2078, US

(73) Assignee: Syracuse University, Syracuse, NY (US)

(21) Appl. No.: 12/471,776

(22) Filed: May 26, 2009

Related U.S. Application Data
(60) Provisional application No. 61/055,703, filed on May 23, 2008.

(51) Int. Cl. C23C 16/44; C07C 43/12; C07D 407/14

(52) U.S. Cl. 427/255.28; 568/677; 549/472

(57) ABSTRACT

Highly volatile MOCVD (Metal-Organic Chemical Vapor Deposition) precursors are disclosed comprising a complex between a fluoroalkoxide ligand and one or more alkali, alkaline earth, lanthanoids or yttrium metals and one or more donor molecules. In one example, the fluoroalkoxide ligand is perfluoro-tert-butoxide and the complex is a heterobimetallic complex. These MOCVD precursors are highly volatile, non-oligomeric, non-pyrophoric and can be synthesized with high yields. They are ideally suited for MOCVD applications because of their ability to vaporize at low temperatures and at atmospheric pressure thus enabling the deposition of a more uniform and homogeneous metal coating of known stoichiometry on to a substrate.

### CROSS-REFERENCE TO RELATED APPLICATION

This patent application claims priority to a provisional patent application, U.S. Ser. No. 61/055,703 filed May 23, 2008, pursuant to 35 U.S.C. §§111 and 119, the entire contents of said application being incorporated by reference herein.

### BACKGROUND OF THE INVENTION

MOCVD (Metal-Organic Chemical Vapor Deposition) is extensively employed for epitaxial thin film growth of III-V semiconductors and other metal combinations in a variety of microfabrication applications, including the manufacture of sub-micron interconnecting structures within microprocessors as well as the production of superconductors for use in photodetectors or lasers, ferroelectrics, and other electronic applications. In this technique, an inert gas stream containing an MOCVD precursor in the gas phase is passed over a substrate, such as a superconductor wafer, which is heated to a temperature that exceeds the decomposition temperature of the MOCVD precursor. Contact of the vaporized MOCVD precursor stream with the heated substrate induces the pyrolysis of the complex's organic ligand and the subsequent deposition of stoichiometric amounts of the associated metal on to the substrate. By varying the composition of the precursor, the properties of the crystal can be altered at an almost atomic scale. MOCVD therefore permits the growth of high quality, uniform semiconductor layers as thin as 1 nanometer with a crystal structure that is perfectly aligned with that of the substrate.

The quest for novel MOCVD precursors of alkaline earth metal complexes with improved properties is the focus of intense worldwide research. Many of these efforts center upon the production of high quality YBa₂Cu₃O_{7 x1-7} films, but other materials, such as BaTiO₃, and BiSrCaCuO₉ are also highly sought after. A wide variety of ligands such as pyrazolates, cyclopentadienes, β-diketonates, polyethers and phenolates are also being tested for MOCVD applications.

There is therefore an unmet need in the field to discover new MOCVD precursors and associated ligands with improved physical-chemical properties for more uniform and controlled chemical vapor deposition.

### SUMMARY OF THE APPLICATION

The application discloses the synthesis, structure and physical-chemical properties of highly volatile MOCVD precursors comprising fluoroalkoxide ligands in coordination with one or more alkaline metals, alkaline earth metals, lanthanoids or Yttrium. A method of MOCVD using the disclosed compounds is also described.

It should be understood that this application is not limited to the embodiments disclosed in this Summary, and it is intended to cover modifications and variations that are within the scope of those of sufficient skill in the field, and as defined by the claims.

In one embodiment, a composition of matter is disclosed comprising a complex between one or more fluoroalkoxide ligands and one or more metals, the complex being represented by the formula:

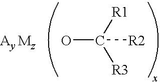

wherein R1, R2 and R3 are fluoroalkyl groups, x, y and z are non-negative integers, wherein y and z are not simultaneously zero, and A and M are metals each selected from the group consisting of alkali metals, alkaline earth metals, lanthanoids and Y.

The complex can be a heterobimetallic complex and non-pyrophoric. Sublimation of this complex can occur at a temperature of at most 240 degrees Celsius at atmospheric pressure.

At least two of the fluoroalkyl groups R1, R2 and R3 can have a different chemical structure from each other. Alternatively, all of the fluoroalkyl groups R1, R2 and R3 can have the same chemical structure.

In another aspect, at least one of the fluoroalkyl groups R1, R2 and R3 comprises a fluorinated methyl group.

In yet another aspect, at least one of the fluoroalkyl groups R1, R2 and R3 is fully fluorinated.

The fluoroalkoxide ligand can be perfluoro-tert-butoxide.

Metal A can be different from metal M. Alternatively, metal A and metal M can belong to the same Group or to different Groups of the Periodic Table.

According to another version, the complex further comprises a plurality of donor molecules. The donor molecules can be selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors. This complex can be non-pyrophoric with an onset of sublimation that occurs at a temperature of at most 240° C. at atmospheric pressure.

In yet another version, the fluoroalkoxide ligand is perfluoro-tert-butoxide and the complex further comprises a plurality of donor molecules. The donor molecules can be selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors. This complex can be non-pyrophoric and the onset of sublimation occurs at a temperature of at most 240° C. at atmospheric pressure.

In one version, a composition of matter is disclosed comprising a complex between one or more fluoroalkoxide ligands and one or more metals, the complex being represented by the formula:

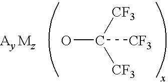

wherein the fluoroalkoxide ligand is perfluoro-tert-butoxide and the metals A and M are each selected from the group consisting of Be, Mg, Ca, Sr and Ba, and x is at least equal to 4, y is equal to 1 and z is equal to 1.

In another version of this complex, the fluoroalkoxide ligand is perfluoro-tert-butoxide and metals A and M are each selected from the group consisting of La, Ce, Pr, Nd, Pm, Eu, Sm, Gd, Tb, Dy, Ho, Er, Tm, Yb, Lu and Y and x is at least equal to 4, 5 or 6, y is equal to 1 and z is equal to 1.

In another version of this complex, the fluoroalkoxide ligand is perfluoro-tert-butoxide and metal A is selected from the group consisting of Be, Mg, Ca, Sr, and Ba and the metal M is selected from the group consisting of Li, Na, K, Rb and Cs, and x is at least equal to 3, y is equal to 1 and z is equal to 1.

In another version of this complex, the fluoroalkoxide ligand is perfluoro-tert-butoxide and metal A is selected from the group consisting of La, Ce, Pr, Nd, Pm, Eu, Sm, Gd, Tb, Dy, Ho, Er, Tm, Yb, Lu and Y and M is a metal selected from the group consisting of Li, Na, K, Rb, and Cs, and x is at least equal to 3 or 4, y is equal to 1 and z is equal to 1.

In yet one version of this complex, the fluoroalkoxide ligand is perfluoro-tert-butoxide and metal A is selected from the group consisting of La, Ce, Pr, Nd, Pm, Eu, Sm, Gd, Tb, Dy, Ho, Er, Tm, Yb, Lu and Y and M is a metal selected from the group consisting of Be, Mg, Ca, Sr and Ba, and x is at least equal to 4 or 5, y is equal to 1 and z is equal to 1.

In one version of this complex, the fluoroalkoxide ligand is perfluoro-tert-butoxide and metal A is selected from the group consisting of La, Ce, Pr, Nd, Pm, Eu, Sm, Gd, Tb, Dy, Ho, Er, Tm, Yb, Lu and Y, y is at least equal to 1, z is equal to zero, and x is at least equal to 2 or 3.

In another version of this complex, the fluoroalkoxide ligand is perfluoro-tert-butoxide and metal A is selected from the group consisting of Be, Mg, Ca, Sr and Ba, y is at least equal to 1, z is equal to zero, and x is at least equal to 2.

In another embodiment, a method for chemical vapor deposition on a substrate is described comprising the steps of: (a) preparing a precursor solution comprising a complex between one or more fluoroalkoxide ligands and one or more metals, the complex being represented by the formula:

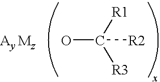

wherein R1, R2 and R3 are fluoroalkyl groups, x, y and z are non-negative integers, wherein y and z are not simultaneously zero, and A and M are metals each selected from the group consisting of alkali metals, alkaline earth metals, lanthanoids and Y, (b) placing the precursor solution in a reactor that is in communication with a substrate, (c) vaporizing the precursor solution to form molecular species in the vapor state; and (d) decomposing the molecular species in the vapor state to deposit a metallic constituent thereof on the substrate, wherein the decomposition of the molecular species in the vapor state on the substrate results in the deposition of one or more metals on the substrate.

In one version of this method, the precursor solution further comprises one or more donor molecules. The donor molecules can be selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

The precursor solution can be non-pyrophoric.

In one aspect, the vaporizing of the precursor solution occurs at atmospheric pressure and without oligomerization.

In one aspect, the decomposition of the molecular species in the vapor state on the substrate results from decomposition of the precursor in contact with the substrate.

In another aspect, the onset of sublimation of the precursor solution can occur at a temperature of at most 240 degrees Celsius at atmospheric pressure. The substrate may comprise a crystalline material or a silicon crystalline material.

In yet another embodiment, a method for chemical vapor deposition on a substrate is described comprising the steps of: (a) preparing a precursor solution comprising a complex between one or more metals and one or more fluoroalkoxide ligands, wherein the complex is represented by the formula:

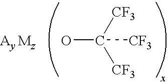

wherein the fluoroalkoxide ligand is perfluoro-tert-butoxide and x, y and z are non-negative integers, wherein y and z are not simultaneously zero, and A and M are metals each selected from the group consisting of alkali metals, alkaline earth metals, lanthanoids and Y, (b) placing the precursor solution in a reactor that is in communication with a substrate, (c) vaporizing the precursor solution to form molecular species in the vapor state; and (d) decomposing the molecular species in the vapor state to deposit a metallic constituent thereof on the substrate, wherein the decomposition of the molecular species in the vapor state on the substrate results in the deposition of one or more metals on the substrate.

In one aspect, the precursor solution further comprises one or more donor molecules. The donor molecules may be selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

This complex can be non-pyrophoric.

In one aspect, the vaporizing of the precursor solution occurs at atmospheric pressure and without oligomerization.

In one aspect, the decomposition of the molecular species in the vapor state on the substrate results from decomposition of the precursor in contact with the substrate.

In another aspect, the onset of sublimation of the complex can occur at a temperature of at most 240 degrees Celsius at atmospheric pressure. The substrate may comprise a crystalline material or a silicon crystalline material.

The previously described embodiments have many advantages. Unlike other MOCVD precursors known in the art, the herein described MOCVD precursors are non-pyrophoric and highly volatile at temperatures below 240° C. and at atmospheric pressure. Primarily, this is because the associated carrier ligands incorporate sterically encumbered fluorinated side groups, as well as donor molecules that increase intermolecular F-F repulsion and prevent precursor oligomerization. The higher volatility of these MOCVD precursors at lower temperatures and at atmospheric pressure translates into improved overall MOCVD quality because the precursors and the substrate have a greater thermal stability under these conditions. The rate of the stoichiometric metal deposition on the substrate can be also increased while, at the same time, reducing the residual deposits that may result from the incomplete decomposition of the associated carrier ligand upon contact with the substrate.

### BRIEF DESCRIPTION OF THE DRAWINGS

FIG. 1 depicts a representative structure of compound Ca(PFTB)₂(diglyme)₂ (compound 1) in accordance with a first embodiment;

FIG. 2 depicts a representative structure of Sr(PFTB)₂(diglyme)₂ (compound 2) in accordance with a second embodiment;

FIG. 3 depicts a representative structure of Ba(PFTB)₂(diglyme)₂ (compound 3) in accordance with a third embodiment;

FIG. 4 depicts a representative structure of K(THF)Sr(μ-PFTB)₃(THF)₃ (compound 14) according to a fourth embodiment;

FIG. 5 depicts a representative structure of K(THF)Sr(μ-PFTB)₃(THF)₃ (compound 14) according to a fifth embodiment;

FIG. 6 depicts a TGA overlay of compound 1, (Ca(PFTB)₂(diglyme)₂), compound 2 (Sr(PFTB)₂(diglyme)₂ and compound 3 (Ba(PFTB)₂(diglyme)₂);

FIG. 7 depicts a TGA overlay of Na(THF)Ba(μ-PFTB)₃(THF)₃ (compound 11), K(THF)Sr(μ-PFTB)₃(THF)₃ (compound 14), and K(THF)Ba(μ-PFTB)₃(THF)₃ (compound 15); and

FIG. 8 depicts crystallographic information for compounds 1 (Ca(PFTB)₂(diglyme)₂), compound 3 (Ba(PFTB)₂(diglyme)₂), compound 11 (Na(THF)Ba(μ-PFTB)₃(THF)₃) compound and 14 (K(THF)Sr(μ-PFTB)₃(THF)₃).

### DETAILED DESCRIPTION

Unless defined otherwise, all technical and scientific terms used herein have the same meaning as is commonly understood by one of skill in the art. The following definitions are provided to help interpret the disclosure and claims of this application. In the event a definition in this section is not consistent with definitions elsewhere, the definition set forth in this section will control.

The term “plurality” as used herein refers to a quantity of two or more.

As used herein, the term “fully fluorinated” refers to a fluoroalkyl group in which each available hydrogen atom is replaced by a fluorine atom.

As used herein, “donor groups” refer to hydrocarbon solvents such as alkyl, aryl, ether or amine hydrocarbon solvents. For example, suitable ether solvents are represented by the general formula R1-O—R2, wherein R1 and R2 are preferably independently selected from an alkyl group, an aryl group or an alkoxy group typically containing from 1 to 12 carbon atoms. Preferred donor molecules include, but are not limited to, tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

As used herein, the term “lanthanoid” or Ln (according to IUPAC terminology) is synonymous with the older term “lanthanoid” and refers to the 15 elements with atomic numbers 57 through 71, from lanthanum to lutetium (according to IUPAC terminology).

As used herein, the term “alkali metals” refers to the series of elements comprising Group 1 of the periodic table (according to IUPAC terminology): lithium (Li), sodium (Na), potassium (K), rubidium (Rb), caesium (Cs), and francium (Fr).

As used herein, the term “alkaline earth metals” refers to the series of elements comprising Group 2 of the periodic table (according to IUPAC terminology): beryllium (Be), magnesium (Mg), calcium (Ca), strontium (Sr), barium (Ba) and radium (Ra).

As used herein, Y refers to the transition metal element Yttrium having the atomic number 39.

As used herein, a “metal” of a PFTB complex refers to PFTB complexes with alkali metals, alkaline earth metals, lanthanoids or Yttrium.

As used herein, the term “IUPAC” refers to the International Union of Pure and Applied Chemistry.

As used herein, “PFTB” is the abbreviation for perfluoro-tert-butoxide.

As used herein, the term “Group” refers to the Groups of the Periodic Table as determined by the International Union of Pure and Applied Chemistry.

As used herein, a “non-pyrophoric” substance is a substance that is stable at room temperature and atmospheric pressure and therefore does not ignite spontaneously.

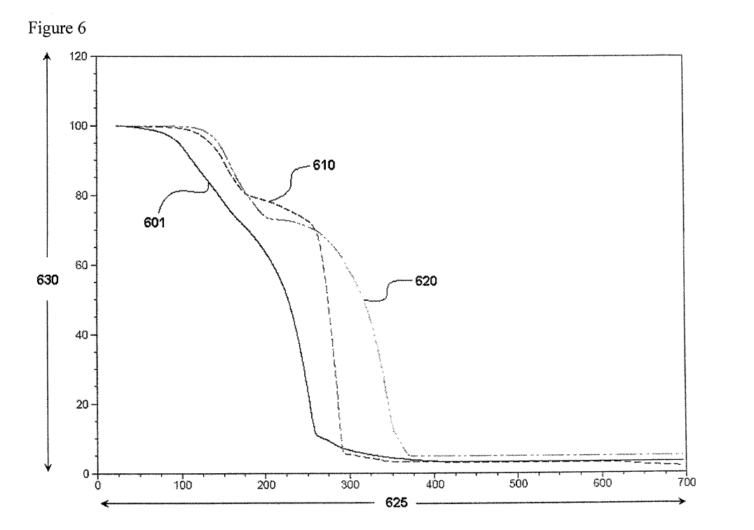

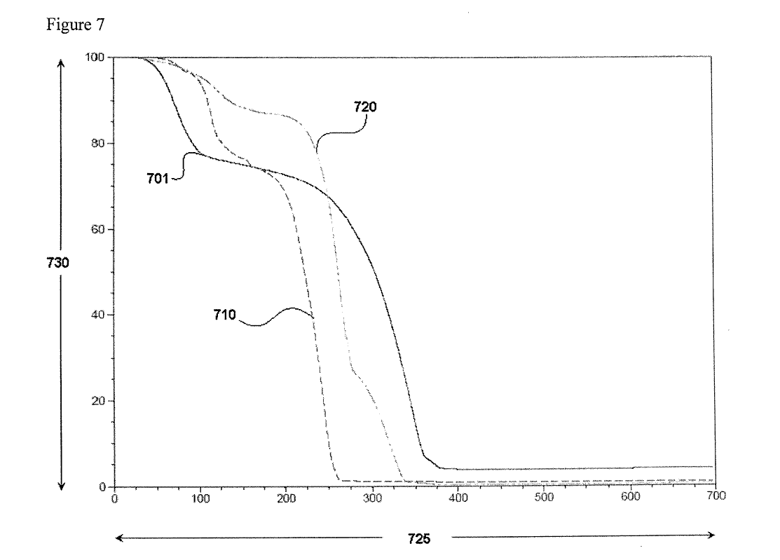

As used herein, the “onset of sublimation” refers to the temperature at atmospheric pressure at which the % weight of a metal fluoroalkoxide complex starts to decrease as a consequence of sublimation i.e. the temperature at which the metal fluoroalkoxide complex transitions from a solid to a gas phase with no intermediate liquid stage. Typically, the onset of sublimation is determined by thermogravimetric (TGA) analysis (for example, see FIGS. 6 and 7). In one embodiment, the onset of sublimation occurs at a temperature of between 500 degrees Celsius and 100 degrees Celsius at atmospheric pressure or between 450 degrees Celsius and 100 degrees Celsius at atmospheric pressure or between 400 degrees Celsius and 100 degrees Celsius at atmospheric pressure or between 350 degrees Celsius and 100 degrees Celsius at atmospheric pressure or between 300 degrees Celsius and 100 degrees Celsius at atmospheric pressure or between 275 degrees Celsius and 100 degrees Celsius at atmospheric pressure or between 250 degrees Celsius and 100 degrees Celsius at atmospheric pressure. In a preferred embodiment, the onset of sublimation occurs at a temperature of between 240 degrees Celsius and 100 degrees Celsius at atmospheric pressure or between 230 degrees Celsius and 100 degrees Celsius at atmospheric pressure or between 220 degrees Celsius and 100 degrees Celsius at atmospheric pressure or between 215 degrees Celsius and 100 degrees Celsius at atmospheric pressure or between 210 degrees Celsius and 100 degrees Celsius at atmospheric pressure or between 200 degrees Celsius and 100 degrees Celsius at atmospheric pressure or between 190 degrees Celsius and 100 degrees Celsius at atmospheric pressure or between 180 degrees Celsius and 100 degrees Celsius at atmospheric pressure or between 170 degrees Celsius and 100 degrees Celsius at atmospheric pressure or between 160 degrees Celsius and 100 degrees Celsius at atmospheric pressure or between 150 degrees Celsius and 100 degrees Celsius at atmospheric pressure or between 140 degrees Celsius and 100 degrees Celsius at atmospheric pressure or between 130 degrees Celsius and 100 degrees Celsius at atmospheric pressure or between 120 degrees Celsius and 100 degrees Celsius at atmospheric pressure or between 110 degrees Celsius and 100 degrees Celsius at atmospheric pressure.

As used herein the term “substrate” refers to any support structure upon which an MOCVD precursor decomposes on contact to deposit stoichiometric amounts of the associated metals. The substrate can be made of any substance provided it can sustain the high temperatures required for MOCVD precursor pyrolysis. In one example, a substrate can be a base wafer comprising silicon or germanium.

As used herein, “atmospheric pressure” is equal to 1 atmosphere=760 mmHg=29.92 in Hg=14.7 lb/in² (psi)=101.3 KPa=760 Torr.

As used herein, the term “oligomerization” refers to the aggregation of MOCVD precursors. In the absence of highly volatile carrier ligands such as PFTB and the like as described herein, MOCVD precursors comprising alkaline and rare earth metals are known to oligomerize thereby greatly reducing the volatility of these MOCVD precursors at atmospheric pressure. Under these circumstances, the gaseous phase can only be attained by submitting the MOCVD precursors to high temperatures and pressures of 10⁻⁵ Torr or less.

With the preceding definitions as noted herein, the following description relates to certain preferred embodiments of the application, and to certain highly volatile MOCVD precursors comprising metallic fluoroalkyloxides precursors as described herein and methods of use.

Fluorocarbons exhibit very different properties compared to their hydrocarbon analogues. The enhanced strength of the C—F over the C—H bond (C—H: 411 kJ mol⁻¹; C—F: 485 kJ mol⁻¹) leads toward greater thermal stability, the multiple non-bonding p-electrons shield the carbon backbone, and the presence of strong electron withdrawing groups adds important inductive effects to the molecule. Therefore, fluoroalcohols are attractive for MOCVD applications because 1) increasing amounts of fluorine increase intermolecular repulsions, and 2) the reduced polarizability of fluorine causes fewer attractive intermolecular interactions. Aside from suppressing aggregation tendencies the fluorinated ligand is capable of intramolecular M•••F contacts that partially satisfies the metals valence, further decreasing oligomerization and possibly providing greater stabilization of the solid while contributing to a greater propensity to volatize at atmospheric pressure. As will be readily apparent from this disclosure, the inventive concepts described herein can also be suitably applied to other methods and compositions that are related to the field of highly volatile MOCVD fluoroalkoxide precursors.

According to a first embodiment, a variety of alkaline earth metal monometallic complexes are disclosed that are synthesized by treatment of alkaline earth hexamethyldisilazides (hexamethyldisilazane=H[HMDS]) and H (PFTB) or (NH₄)(PFTB)) in ethereal solution. This transamination method at room temperature takes advantage of the low pKa of H(PFTB) (pKa=5.2) and the high pKa of H(HMDS) (pKa≈30) to drive the reaction forward.

In Synthesis Scheme 1, the perfluoro-tert-butoxide (PFTB) precursors comprising alkaline earth (Ae) metals are synthesized as follows:

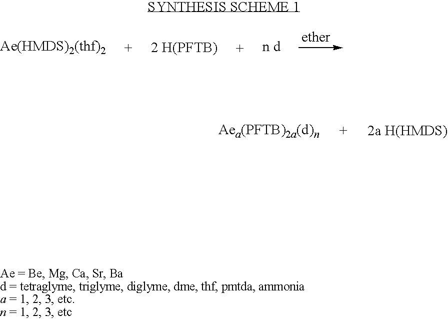

In Synthesis Scheme 2, the perfluoro-tert-butoxide (PFTB) precursors comprising alkaline earth metals (Ae) are synthesized in the presence of solid ammonium PFTB as follows:

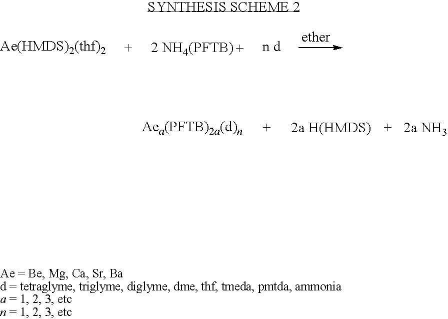

Both routes provide alkaline earth complexes of varying metal stoichiometries with differing amounts of donor and/or ammonia coordination in excellent yields and quality.

According to a second embodiment, synthetic protocols based on direct metallation are disclosed for the synthesis of analogous alkaline earth metal complexes utilizing ammonia chemistry. Condensation of anhydrous ammonia into the solutions containing alkaline earth metals with either H(PFTB) or (NH₄)(PFTB) under reflux conditions (see Synthesis Schemes 3 and 4 below) yields analogous compounds to those described in Synthesis Schemes 1 and 2.

In direct metallation Synthesis Scheme 3, the perfluoro-tert-butoxide (PFTB) precursors comprising alkaline earth (Ae) metals are synthesized in the presence of H(PFTB) as follows:

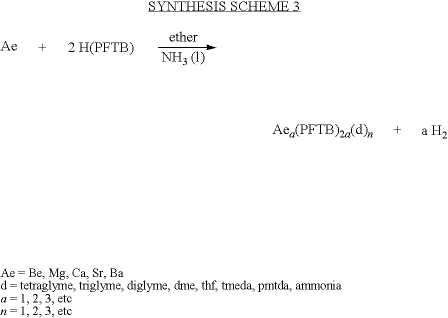

In direct metallation Synthesis Scheme 4, the perfluoro-tert-butoxide (PFTB) precursors comprising alkaline earth (Ae) metals are synthesized in the presence of NH₄(PFTB as follows:

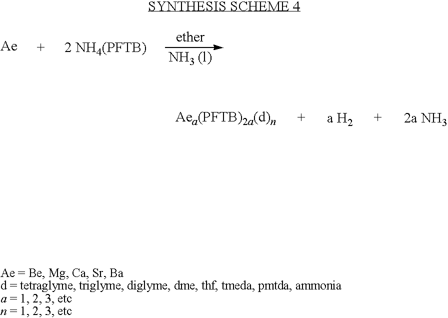

The compounds resulting from Synthesis Schemes 1-4 include donor molecules as defined herein such as tetraglyme, triglyme, diglyme, dimethoxyethane (DME), tetrahydrofuran (THF), N,N,N,′N′,′N″-pentamethyltriethylenediamine (PMTDA), and N,N,N,′N′-tetramethlyethylenediamine (TMEDA). They can be easily purified by crystallization from hexane layered ether solutions at a variety of temperatures ranging from −23° C. to room temperature.

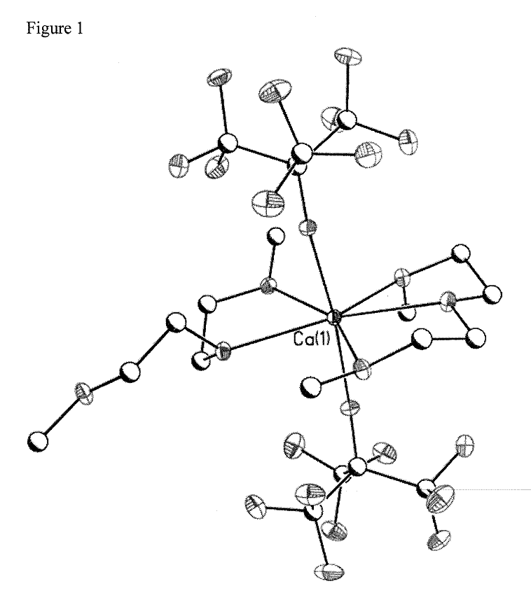

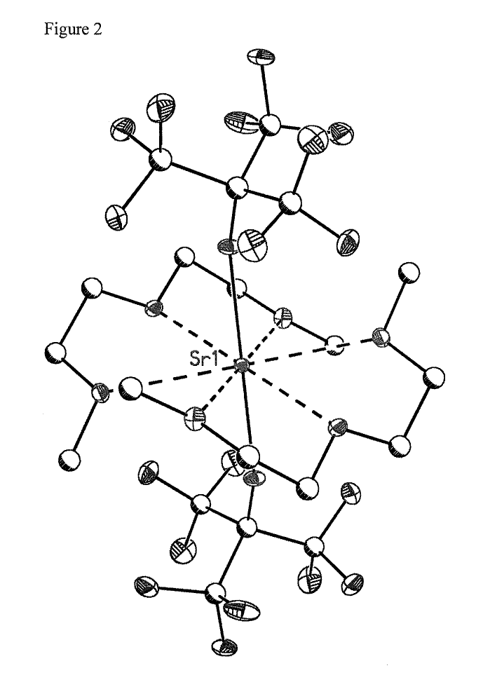

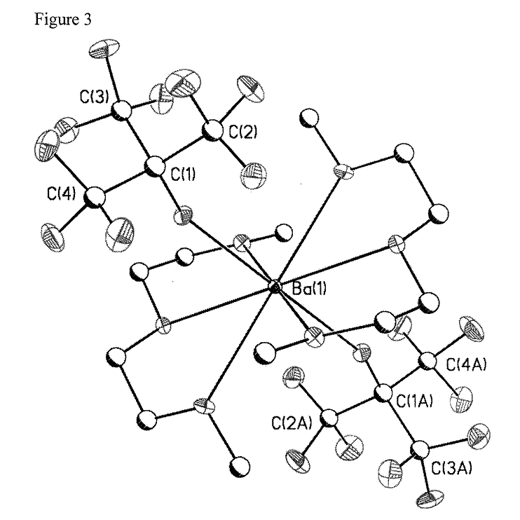

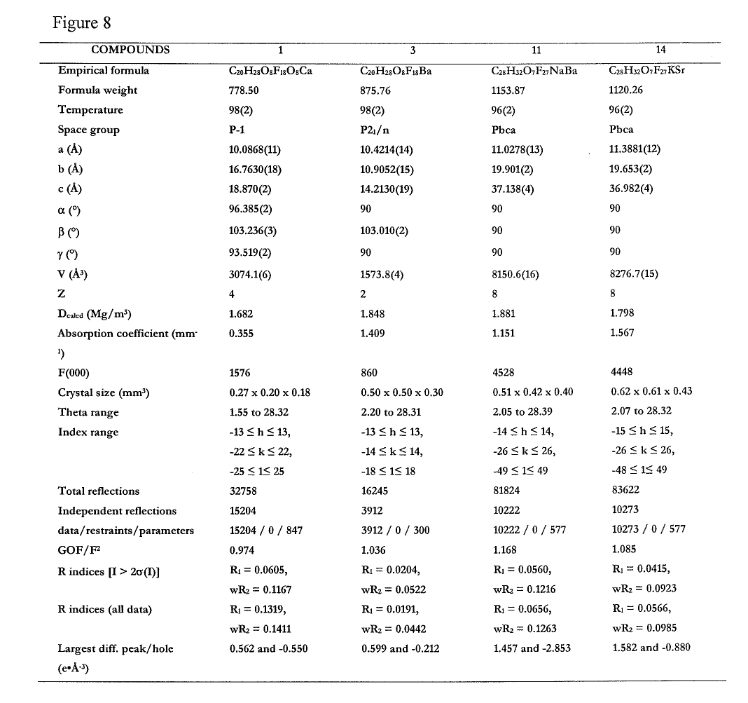

The monometallic PFTB complexes made from Synthesis Schemes 1-4 were analyzed by crystallography (see Tables 1, 2 and FIG. 8) and found to have the general formula Ae (PFTB)₂(d)ₙ (Ae=Be, Mg, Ca, Sr, Ba; d=tetraglyme, triglyme, diglyme, DME, THF, TMEDA, PMTDA, ammonia). Specifically compounds of the formula Ae(PFTB)₂(diglyme)₂ (Ae=Ca, (compound 1); Sr, (compound 2); Ba, (compound 3)) are described in detail below. The structures of compounds 1-3 all follow the same structural trend where PFTB occupies the apical positions and donors are arranged in the equatorial positions. Representative examples of compounds 1, 2 and 3 are depicted in FIG. 1, FIGS. 2 and 3 respectively (all atoms, except for carbon, shown as ellipsoids at 30% probability. Hydrogen atoms are omitted for clarity). The equatorial diglymes deviate moderately from the equatorial plane with differences attributed to non-standard C—F•••H—C interactions.

Referring to FIG. 1, compound 1 has several unique features. Two crystallographically unique but similar calcium environments exist within the unit cell. Both calcium atoms display coordination numbers of seven with severely distorted pentagonal bipyramid geometries. The fluoroalkoxides occupy the axial positions, with the diglyme donors filling the equatorial positions. The two diglyme donors coordinate differently to the metal center, one is a tridentate donor, while the second coordinates in a bidentate mode. The O_{L}-M-O_{d} angles ranged from 86.37(7)° to 102.37(7)° on Ca (1) and 85.13(7)° to 101.50(8)° on Ca (2) forming a tilted ring around the central metal. The O_{L}-M-O_{L} angle varied moderately [(172.51(8) Ca (1) and 174.73(8) Ca (2)] for each calcium. Bent ligand angles exist for many alkaline earth metal complexes. Bending energies as little as 1.5 kJ mol⁻¹ for the cyclopentadienyl barium compounds demonstrate the flexibility of alkaline earth complexes. Although, bending effects typically become more exaggerated with the heavier conjurers of the series, both compounds 2 and 3 show a linear trans angle.

Referring to FIGS. 2 and 3, in compounds 2 and 3, the coordinated diglymes in the equatorial plane display deviations of the O_{L}-M-O_{d} angles between non-symmetry generated atoms vary between 86.31(5)-102.79(4)° for compound 2 and 89.28(3)-104.13(4)° for compound 3. This results in significant deviation from orthogonal geometry; the equatorial plane tilts to a maximum of 12.8° for compound 2 and 14.1° for compound 3, illuminating the structural similarities between the two compounds. Each metal center shows an eight coordinate metal center with six donor atoms in the equatorial plane, in addition to two trans interactions from the ligand. In contrast to compound 1, the two coordinating diglymes are both tridentate for 2 and 3.

As shown in Table 1, an extensive network of non-traditional C—F•••H—C interactions are observed in compounds 1-3. These interactions, while individually relatively weak, compared to the F•••H—O hydrogen bond (20-40 kJ mol⁻¹), may provide significant stabilization. Each non-traditional C—F•••H—C contact adds 4.5 kJ mol⁻¹(d(H•••F)=2.6 Å) of stabilizing energy to the molecule. The summation of C—F•••H—C interaction may rationalize the different geometry for compound 1 compared to 2 and 3. The calcium complex forms the most secondary interaction within the family. A total of ten intra- and six intermolecular interactions appear for Ca(1) and nine intra- and six intermolecular interactions for Ca(2). The average intramolecular C—F•••H—C distances were shorter than the average intermolecular C—F•••H—C distances for compound 1 (intramolecular: average 2.56 Å, shortest 2.514 Å, longest 2.689 Å; intermolecular: average 2.62 Å, shortest 2.300 Å, longest 2.697 Å). Compound 2 showed eight intramolecular C—F•••H—C contacts with average lengths of 2.4 Å, and two or four intermolecular C—F•••H—C contacts (depending on disorder) with mean distance of 2.5 Å. Compound 3 yielded four intermolecular interactions for each disordered position (average 2.6 Å) and only six intramolecular interactions for each disordered position (average 2.6 Å). For the non-traditional C—F•••H—C interactions cutoff limits were based on the sum of the Van der Waals interactions. Only contacts with distance shorter than 2.7 Å were included.

In analogy with related species, the fluoromethyl groups show rotational disorder. This disorder was observed in compounds 2 and 3, but was successfully refined using split positions. Compound 2 refined successfully with a 12/78, 20/80, 19/81 occupancy for C2, C3, C4 and a 47/53, 42/58, 48/52 occupancy for C2, C3, C4 in compound 3. All three fluoroalkoxide monometallic compounds had, relatively, the same average C—F distances (1: 1.34 Å; 2: 1.34 Å; 3: 1.33 Å) and the same short C—OL distances (1: average 1.33 Å; 2: 1.323(2) Å; 3: 1.327(2) Å).

TABLE 1
Inter- and Intramolecular C-F•••H-C distances from compound 1.

| | C-F•••H-C | | C-F•••H-C |
|---|---|---|---|
| Ca(1) | (inter-) Å | Ca(2) | (inter-) Å |
| F(1)-H(20A) | 2.689 | F(22)-H(31A) | 2.591 |
| F(4)-H(16A) | 2.679 | F(24)-H(33B) | 2.546 |
| F(7)-H(31A) | 2.662 | F(27)-H(19B) | 2.652 |
| F(14)-H(19A) | 2.651 | F(30)-H(37B) | 2.524 |
| F(18)-H(11A) | 2.672 | F(31)-H(16B) | 2.514 |
| F(18)-H(12B) | 2.576 | F(32)-H(40B) | 2.673 |
| Average | 2.62 | | |
| F(3)-H(9C) | 2.697 | F(20)-H(36A) | 2.644 |
| F(6)-H(13A) | 2.541 | F(20)-H(35B) | 2.529 |
| F(7)-H(18B) | 2.635 | F(21)-H(38A) | 2.582 |
| F(8)-H(16B) | 2.687 | F(23)-H(32A) | 2.526 |
| F(8)-H(15C) | 2.479 | F(24)-H(35B) | 2.559 |
| F(12)-H(14C) | 2.569 | F(26)-H(31B) | 2.489 |
| F(13)-H(17B) | 2.595 | F(29)-H(34B) | 2.377 |
| F(14)-H(9B) | 2.618 | F(35)-H(29C) | 2.300 |
| F(16)-H(17B) | 2.577 | F(36)-H(37A) | 2.695 |
| F(17)-H(14C) | 2.634 | | |
| Average | 2.56 | | |

TABLE 2
Comparison of inter- and intramolecular C-F•••H-C
contact distances for compounds 2 and 3.

| Compound | C-F•••H-C (inter-) Å | | C-F•••H-C (intra-) Å | |
|---|---|---|---|---|
| Sr(PFTB)₂(diglyme)₂ | F(4)-H(5A) | 2.601 | F(1)-H(5B) | 2.256 |
| (compound 2) | F(8)-H(9A) | 2.352 | F(2)-H(8BA) | 2.536 |
| | F(4A)-H(5AA) | 2.601 | F(8)-H(9BA) | 2.482 |
| | F(8A)-H(9AA) | 2.352 | F(9)-H(5B) | 2.584 |
| | | | F(1A)-H(5BA) | 2.256 |
| | | | F(2A)-H(8B) | 2.536 |
| | | | F(8A)-H(9B) | 2.482 |
| | | | F(9A)-H(5BA) | 2.584 |
| Average | | 2.5 | | 2.4 |
| Ba(PFTB)₂(diglyme)₂ | F(1′)-H(5A) | 2.672 | F(3′)-H(8C) | 2.560 |
| (compound 3) | F(8′)-H(5A) | 2.594 | F(4′)-H(5BA) | 2.605 |
| | F(1′A)-H(5AA) | 2.672 | F(7′)-H(8B) | 2.560 |
| | F(8′A)-H(5AA) | 2.594 | F(3′)-H(8CA) | 2.560 |
| | | | F(4′)-H(5B) | 2.605 |
| | | | F(7′A)-H(8BA) | 2.560 |
| Average | | 2.6 | | 2.6 |

Only contacts resulting from one disordered position are shown.

According to a third embodiment, a variety of lanthanoid monometallic complexes are disclosed that are also synthesized using transamination chemistry (see Synthesis Schemes 5 and Scheme 6). Utilization of lanthanoid hexamethyldisilazides (hexamethyldisilazane ═H[HMDS]) and either H(PFTB) or (NH₄)(PFTB) in ether solutions yields lanthanoid complexes of varying stoichiometries with differing amounts of donor and/or ammonia coordination. For the transamination route, the oxidation state of resulting complexes is controlled by the oxidation state of the lanthanoid hexamethyldisilazide starting materials.

In Synthesis Scheme 5, the perfluoro-tert-butoxide (PFTB) precursors comprising lanthanoid (Ln) metals are synthesized in the presence of H(PFTB). The chemical reaction of Synthesis Scheme 5 can be summarized as follows:

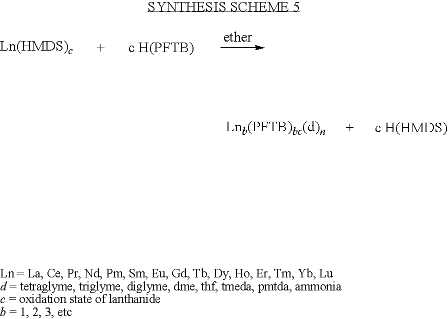

In Synthesis Scheme 6, the perfluoro-tert-butoxide (PFTB) precursors comprising lanthanoid (Ln) metals are synthesized in the presence of solid ammonium PFTB. The chemical reaction of Synthesis Scheme 6 can be summarized as follows:

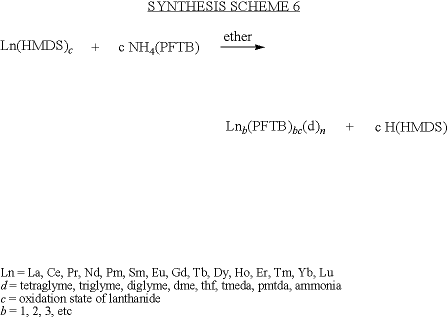

According to a fourth embodiment, the synthesis of analogous lanthanoid metal complexes is described using ammonia chemistry. Condensing dry ammonia into ether solutions of lanthanoid metal with either H(PFTB) or (NH₄)(PFTB) followed by condensation of ammonia and subsequent reflux (see Synthesis Schemes 7 and Scheme 8 below) to yield the lanthanoid PFTB complexes analogous to those obtained in Scheme 5 and Scheme 6.

In direct metallation Synthesis Scheme 7, the perfluoro-tert-butoxide (PFTB) precursors comprising Lanthanoid (Ln) metals are synthesized in the presence of H(PFTB) as follows:

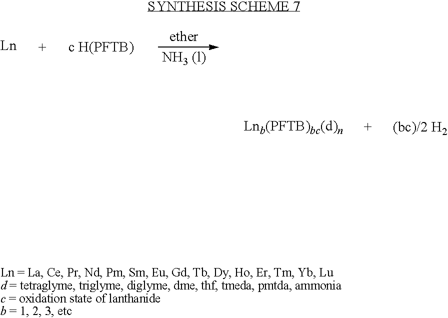

In direct metallation Synthesis Scheme 8, the perfluoro-tert-butoxide (PFTB) precursors comprising Lanthanoid (Ln) metals are synthesized in the presence of (NH₄)(PFTB) as follows:

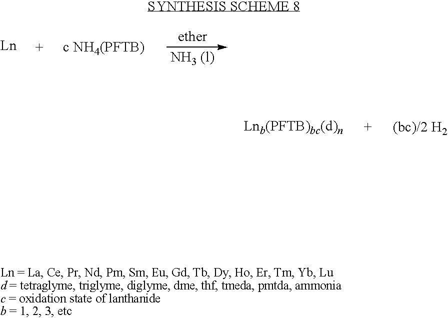

The compounds resulting from Schemes 5-8 include donor molecules as defined herein such as tetraglyme, triglyme, diglyme, dimethoxyethane (DME), tetrahydrofuran (THF), N,N,N,′N′,′N″-pentamethyltriethylenediamine (PMTDA), and N,N,N,′N′-tetramethlyethylenediamine (TMEDA). Purification may easily achieved by crystallization from hexane layered ether solutions at a variety of temperatures ranging from −23° C. to room temperature.

According to a fifth embodiment, alkali (A)/alkaline earth (Ae) heterobimetallic fluoroalkoxides are prepared by combining alkali and alkaline earth monometallic PFTB complexes according to synthesis schemes 9 and 10 below. The syntheses use an alkali hydride with either H(PFTB) or (NH₄)(PFTB) to yield the corresponding alkali PFTB complex. The alkali PFTB component is then combined with the alkaline earth PFTB complex, as described in synthesis schemes 1-4 (see above), to produce heterobimetallic compounds. Pure crystalline compounds were obtained by crystallization from ethereal solutions layered with hexane at a variety of temperature ranging from −23° C. to room temperature.

In Synthesis Scheme 9, the perfluoro-tert-butoxide (PFTB) precursors comprising alkali metals are first synthesized in the presence of H(PFTB) and then combined with the Aeₐ(PFTB)₂ₐ(d)ₙ produced by synthesis schemes 1-4 as follows:

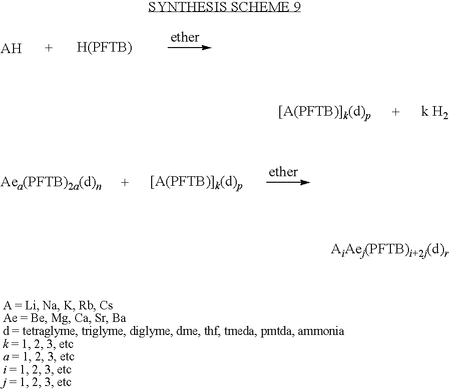

In Synthesis Scheme 10, the perfluoro-tert-butoxide (PFTB) precursors comprising alkali metals are first synthesized in the presence of NH₄(PFTB) and then combined with the Aeₐ(PFTB)₂ₐ(d)ₙ produced by synthesis schemes 1-4. The chemical reactions of Synthesis Scheme 10 can be summarized as follows:

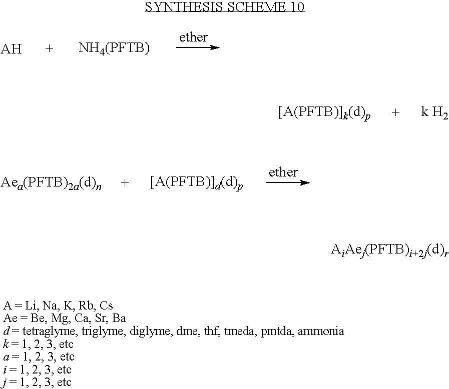

The alkali/alkaline earth heterobimetallic fluoroalkoxides made from Synthesis Schemes 9-10 were analyzed by crystallography (see Table 3 and FIG. 8). Illustrative examples of the alkali/alkaline earth heterobimetallic fluoroalkoxides include Na(THF)Ba(μ-PFTB)₃(THF)₃ (compound 11), K(THF)Sr(μ-PFTB)₃(THF)₃ (compound 14), and K(THF)Ba(μ-PFTB)₃(THF)₃ (compound 15).

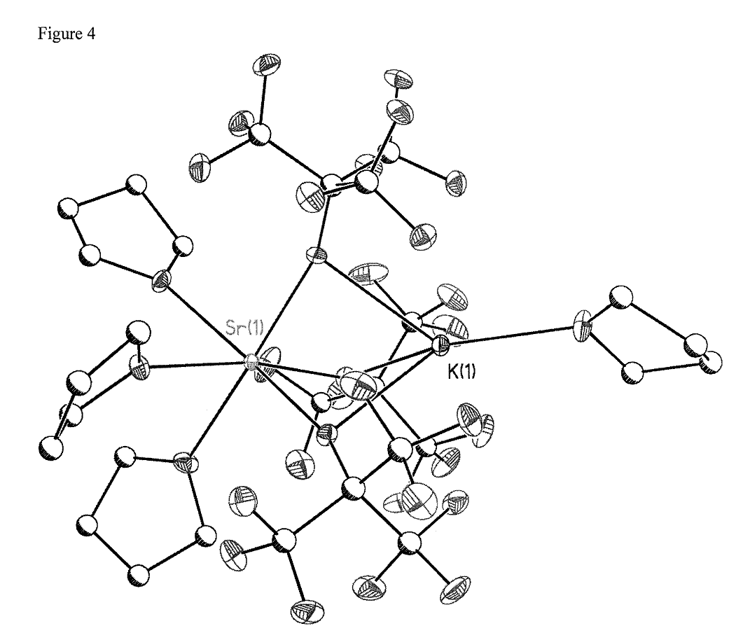

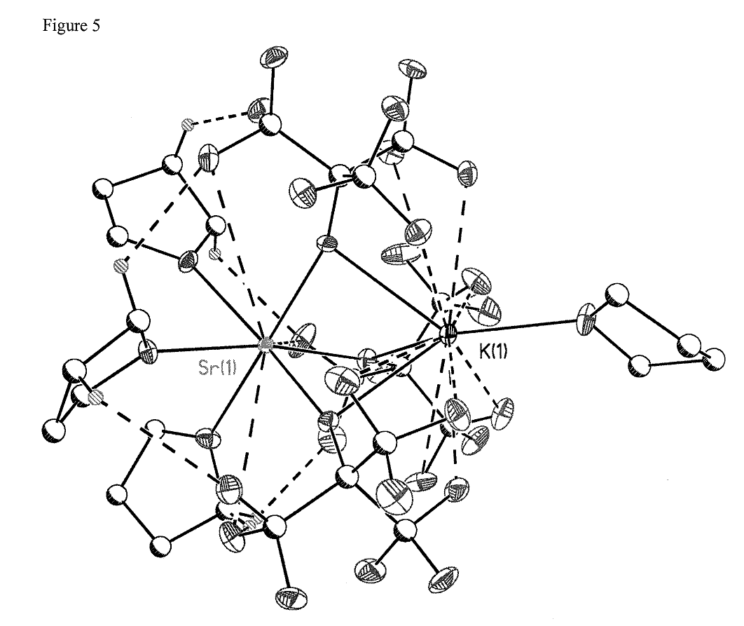

The structure of compound 14 is shown in FIG. 4. FIG. 5 depicts the structure of compound 14 with secondary interactions (M•••F and C—F•••H—C) that give thermal stability to the complex. Both compounds 11 and 15 are isostructural. In FIGS. 4 and 5, all atoms except for carbon are shown as ellipsoids at 30% probability. Hydrogen atoms are either omitted for clarity (FIG. 4) or included if they contribute to secondary interactions (FIG. 5).

Referring to Table 3, compounds 11, 14, and 15 display a uniform structural pattern with the two metal centers bridged by three fluoroalkoxides. The coordination environments of the metals are saturated by THF donors in addition to multiple M•••F interactions, leading to coordination numbers of six for the alkaline earth metals and four for the alkali metals. Alkali-alkaline earth heterobimetallic PFTB complexes contain several intermolecular and intramolecular C—F•••H—C interactions (see Tables 2 and 3). Four intra-(average 2.6 Å) and five intermolecular (average 2.6 Å) interactions were attained for compound 11; while six intra-(average 2.6 Å) and 10 intermolecular (average 2.6 Å) were observed in compound 14. However, several significant intramolecular M•••F interactions are observed within the sum of Van der Waals radii. Six Na•••F interactions averaging 3.1 Å, and five Ba•••F contacts (average 3.6 Å) were observed for compound 1. Remarkably, compound 14 contains nine 1K•••F (average 3.28 Å) interactions, but only three Sr•••F (average 3.9 Å) interactions. Compound 11 retains all six Na•••F interactions (<3.8 Å) and three Ba•••F interactions (<3.57 Å). Compound 14 exhibits six K•••interactions (<3.094 Å) but no Sr•••F interactions are observed below the cutoff of 3.140 Å. The C—F bond distances remain within the normal range (compound 11: average 1.33 Å; compound 14: average 1.34 Å), while the fluoroalkoxide O_{L}-C distances were short (compound 11: average 1.34 Å; compound 14: average 1.33 Å), like the monometallic PFTB complexes.

TABLE 3
Inter- and Intramolecular C—F•••H—C and
M•••F contacts in Na(THF)Ba(μ-PFTB)₃(THF)₃ (compound 11)
and K(THF)Sr(μ-PFTB)₃(THF)₃ (compound 14).

| | | F•••H | | F•••H | | |
|---|---|---|---|---|---|---|
| Cmpd. | | (inter-) | | (intra-) | | M•••F |
| 11 | F(4)-H(23B) | 2.668 | F(7)-H(21B) | 2.562 | Na(1)-F(2) | 3.074(4) |
| | F(11)-H(19A) | 2.623 | F(10)-H(13B) | 2.641 | Na(1)-F(4) | 3.390(4) |
| | F(12)-H(17A) | 2.709 | F(17)-H(20B) | 2.641 | Na(1)-F(10) | 2.753(4) |
| | F(14)-H(18A) | 2.521 | F(25)-H(25A) | 2.659 | Na(1)-F(13) | 3.405(4) |
| | F(26)-H(16B) | 2.512 | | | Na(1)-F(19) | 3.439(4) |
| | | | | | Na(1)-F(22) | 2.555(4) |
| | | | | | Ba(1)-F(1) | 4.113(4) |
| | | | | | Ba(1)-F(7) | 3.237(4) |
| | | | | | Ba(1)-F(12) | 3.960(4) |
| | | | | | Ba(1)-F(17) | 3.309(3) |
| | | | | | Ba(1)-F(25) | 3.397(3) |
| Average | | 2.6 | | 2.6 | | 3.1 (Na•••F); |
| | | | | | | 3.6 (Ba•••F) |
| 14 | F(2)-H(19A) | 2.628 | F(1)-H(17B) | 2.656 | K(1)-F(6) | 2.847(2) |
| | F(4)-H(20A) | 2.443 | F(2)-H(24A) | 2.552 | K(1)-F(8) | 3.099(2) |
| | F(7)-H(28B) | 2.631 | F(10)-H(23B) | 2.592 | K(1)-F(9) | 4.065(3) |
| | F(10)-H(22B) | 2.666 | F(11)-H(25A) | 2.646 | K(1)-F(14) | 2.917(2) |
| | F(12)-H(16A) | 2.600 | F(19)-H(26A) | 2.708 | K(1)-F(17) | 3.910(3) |
| | F(12)-H(18B) | 2.566 | F(20)-H(17A) | 2.453 | K(1)-F(18) | 2.963(2) |
| | F(15)-H(27B) | 2.550 | | | K(1)-F(24) | 2.861(2) |
| | F(21)-H(15B) | 2.651 | | | K(1)-F(26) | 2.931(2) |
| | F(24)-H(26B) | 2.701 | | | K(1)-F(27) | 3.891(3) |
| | F(25)-H(27A) | 2.645 | | | Sr(1)-F(2) | 3.759(2) |
| | | | | | Sr(1)-F(11) | 3.901(2) |
| | | | | | Sr(1)-F(20) | 3.932(2) |
| Average | | 2.6 | | 2.6 | | 3.28 (K•••F); |
| | | | | | | 3.9 (Sr•••F) |

All the contacts with the Van der Waals sum are shown. Distances are shown in Angstroms (Å).

Crystallographic data obtained for the K(THF)Ba(μ-PFTB)₃(THF)₃ (compound 15) showed complex disorder, involving rotational disorder of the fluoromethyl groups in addition to uncertainty with metal positions. Disorder between K/Ba has been disclosed previously and can be attributed to the similar ionic radii of Ba and K (Ba: 1.35 Å; K: 1.38 Å). Consequently, structural details of K(THF)Ba(μ-PFTB)₃(THF)₃ (compound 15) are not shown.

According to a sixth embodiment, alkali/rare earth heterobimetallic fluoroalkoxide complexes are prepared using protocols that are similar to those described for the synthesis of alakali/alkaline earth metal fluoroalkoxide described above.

In Synthesis Scheme 11, alkali PFTB complexes are first synthesized by reacting alkali hydride with either H(PFTB). The alkali PFTB product is then combined with the rare earth PFTB complex, synthesized according Synthesis Schemes 5-8, to yield alkali/rare earth heterobimetallic fluoroalkoxide complexes. The chemical reactions of Synthesis Scheme 11 can be summarized as follows:

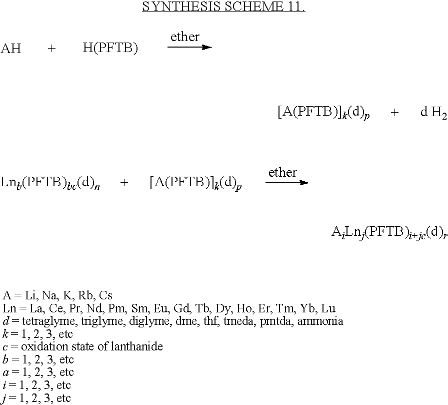

Alternatively, alkali PFTB complexes can be synthesized by reacting an alkali hydride with (NH₄)(PFTB). The alkali PFTB product is then combined with the rare earth PFTB complex, synthesized according Synthesis Schemes 5-8, to yield alkali/rare earth heterobimetallic fluoroalkoxide complexes. The chemical reactions of Synthesis Scheme 12 can be summarized as follows:

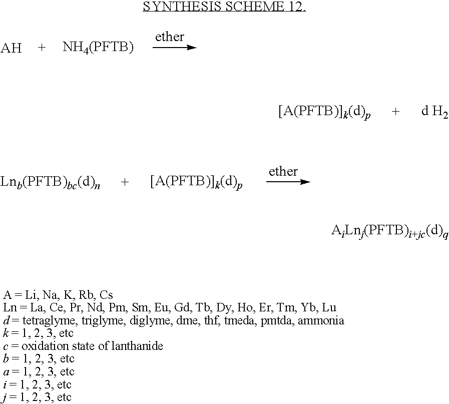

According to a sixth embodiment, alkaline earth/alkaline earth heterobimetallic fluoroalkoxide complexes are prepared in accordance with Synthesis Scheme 13 by combining two different alkaline earth PFTB complexes synthesized in an ethereal solution as described in Synthesis Schemes 1-4. The chemical reaction of Synthesis Scheme 13 can be summarized as follows:

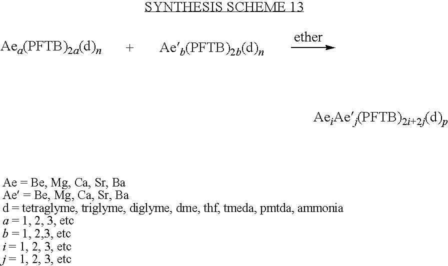

Pure crystalline compounds may be obtained after workup of the solution and crystallization from ethereal solutions layered with hexane at a variety of temperatures ranging from −23° C. to room temperature.

According to a seventh embodiment, rare earth/alkaline earth heterobimetallic fluoroalkoxide complexes are prepared in accordance with Synthesis Scheme 14 by combining an alkaline earth and a rare earth PFTB complex in an ethereal solution. Their individual preparations is outlined in Synthesis Schemes 1-8 described herein. The chemical reaction of Synthesis Scheme 14 can be summarized as follows:

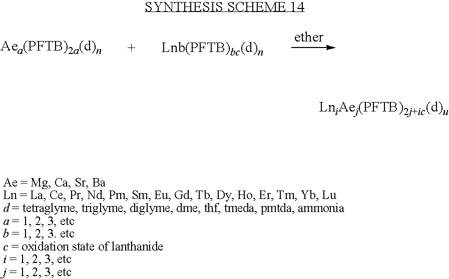

Pure crystalline compounds may be obtained after workup of the solution and crystallization from ethereal solutions layered with hexane at a variety of temperatures ranging from −23° C. to room temperature.

According to an eighth embodiment, rare earth/rare earth heterobimetallic fluoroalkoxides are prepared by combining two different rare earth PFTB complexes in an ethereal solution. The rare earth PFTB complexes are synthesized according to the herein described Synthesis Schemes 5-8. The chemical reaction of Synthesis Scheme 15 can be summarized as follows:

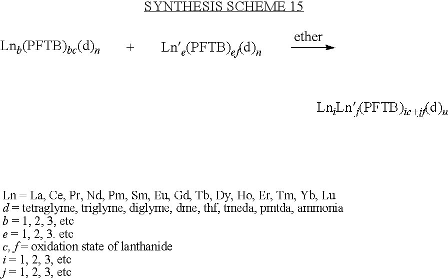

Pure crystalline compounds were obtained after workup of the solution and crystallization from ethereal solutions layered with hexane at a variety of temperatures ranging from −23° C. to room temperature.

With the foregoing description of the synthesis schemes and structures of the PFTB ligand complexed with alkali metals, alkaline earth metals, lanthanoids and Yttrium. thermogravimetric analyses (TGA) of the isolated PFTB metal complexes are described and summarized in Table 4 below.

TABLE 4
TGA and Sublimation data for alkaline earth MOCVD precursors.

| | TGA | | |
|---|---|---|---|
| | T^{a} | T₀^{b} | |
| Compound | (° C.) | (° C.) | % wt. |
| Li(PFTB) | | | |
| Na(PFTB) | | | |
| K(PFTB) | | | |
| Ca(PFTB)₂ | | | |
| Ca(PFTB)₂(diglyme)₂ (compound 1) | 175* | 400 | 3.3-6.6 |
| Sr(PFTB)₂ | | | |
| Sr(PFTB)₂(diglyme)₂ (compound 2) | 175 | 290 | 1.4-4 |
| K(THF)Sr(μ-PFTB)₃(THF)₃ (compound 14) | 160 | 268 | 1.3-1.7 |
| Ba(PFTB)₂ | | | |
| Ba(PFTB)₂(diglyme)₂ (compound 3) | 210 | 370 | 5-17 |
| Na(THF)Ba(μ-PFTB)₃(THF)₃ (compound 11) | 220 | 390 | 4.0-4.4 |
| K(THF)Ba(μ-PFTB)₃(THF)₃ (compound 15) | 210 | 340 | 1.2-3.3 |

^{a}Sublimation onset temperature
^{b}Sublimation complete temperature
*Estimated sublimation onset, the weight percent change for Ca(PFTB)₂(diglyme)₂ (compound 1) gradually decreases.

A person of skill in the art will recognize that MOCVD precursors, such as the copper fluoroalkoxide compounds described in Purdy et al. (U.S. Pat. No. 5,306,836), require a significant vacuum (10⁻⁵ Torr) in order to sublime at sufficiently low temperatures for MOCVD applications. On the contrary, the PFTB complexes described herein, all sublimed at comparatively lower temperatures and at atmospheric pressure. Furthermore, the coordinated donors result in a significant decrease in the sublimation point of these complexes.

Referring to the thermogravimetric analysis (TGA) overlay of FIG. 6, the % weight (630) of the monometallic PFTB compounds 1-3 (601, 610 and 620 respectively) are depicted as a function of increasing temperature in degrees Celsius (625). Compounds 1-3 show clean sublimation by TGA analysis with the exception of compound 1. Compound 1 changed weight percent by gradual decreases, indicating the complex decomposes before or during sublimation. Thermal gravimetric analysis of compounds 2 and 3, however, show loss of the coordinated diglymes before sublimation of the complexes. Sublimation onset occurs at 175° C. and 210° C. with sublimation completion at 290° C. and 370° C. for compounds 2 and 3, respectively.

Referring to the TGA overlay of FIG. 7, the % weight (730) of the heterobimetallic PFTB compounds 11, 14 and 15 (701, 710 and 720 respectively) is depicted as a function of increasing temperature in degrees Celsius (725). Compounds 11, 14, and 15 all show a lower sublimation onset and completion temperatures, in addition to consistently lower percent weights than their monometallic counterparts. The Ba complexes (compounds 11 and 15) show great improvement compared to the TGA profile of compound 3; compounds 11 and 15 have comparable sublimation onset and completion temperatures however, the weight percents are significantly lower. Compound 14 also shows improvement over its monometallic counterpart, compound 2; lower sublimation onset and completion temperatures, with a significantly decreased weight percent. Compounds 11, 14, and 15 all show clean TGAs, where initially the four coordinated THF molecules sublime; followed by sublimation onset at 220° C., 160° C., 210° C. for compounds 11, 14, and 15, and sublimation completion at 390° C., 268° C., 340° C. for compounds 11, 14, and 15, respectively.

In accordance with a ninth embodiment, a MOCVD process is now described for the deposition of volatile metal PFTB precursor complexes of the present application on a suitable substrate. Methods of MOCVD are well known in the art (for example, U.S. Pat. No. 6,887,523, the contents of which are hereby incorporated herein in its entirety). For example, a heterobimetallic PFTB MOCVD precursor described herein is first heated to induce vaporization/sublimation at atmospheric pressure and then transported into a reaction chamber as part of a carrier inert gas flow. The temperature of vaporization/sublimation of the heterobimetallic PFTB MOCVD precursors is lower than the typical MOCVD precursor known in the art (at atmospheric pressure). The gas mixture then flows into a reactor chamber at atmospheric pressure where a substrate such as a silicon wafer is heated with, for example, resistance heaters to a temperature that exceeds the decomposition temperature of the selected MOCVD precursor. Decomposition temperatures at atmospheric pressure may be from 1200° C. to 100° C. or 1100° C. to 100° C. or 1000° C. to 100° C. or 900° C. to 100° C. or 800° C. to 100° C. or 700° C. to 100° C. or 600° C. to 100° C. In a preferred embodiment, the MOCVD decomposition temperature can be from 500° C. to 100° C. or 450° C. to 100° C. or 400° C. to 100° C. or 350° C. to 100° C. or 300° C. to 100° C. or 250° C. to 100° C. or 200° C. to 100° C. or 150° C. to 100° C. Decomposition of the reactive gases upon contact with the substrate leads to the deposition of thin epitaxial layers of the associated metals i.e. alkali metals, alkaline earth metals, Yttrium or lanthanoids or combinations thereof as described herein. The thickness of the layers can range from a few nanometers to a few microns thick, as required. In one example, a state-of-the-art MOCVD reactor can accommodate 50, 75 or 100 or more substrates. Substrates may be circular or square or may have any shape or dimension depending on their intended use.

The invention will now be further illustrated with reference to the following examples. It will be appreciated that what follows is by way of example only and that modifications to detail may be made while still falling within the intended scope of the invention.

### EXAMPLES

All compounds were handled using modified Schlenk techniques with either purified Ar or N₂ atmospheres and special concerns for limiting exposure to water and oxygen. Alkali hydrides were washed three times with hexane, dried and store in a glove box. Potassium and sodium metal were also washed three times with hexane, and then stored in hexane in a glove box. PFTB was refluxed with a dry ice condenser and distilled from calcium hydride and stored in a Schlenk at −13° C. Diglyme was refluxed over calcium hydride and vacuum distilled prior to use. The aryl ligand, 2-phenylphenol was commercially available and used as received. Solvents were collected from a solvent purification system and degassed with three freeze/thaw cycles before use. IR spectra were collected using the Nicolet L200 FTIR spectrometer over the range of 4000 to 400 cm⁻¹. IR samples were prepared using mineral oil mulls sandwiched between KBr plates. ¹H, ¹³C, and ¹⁹F NMR spectra collected using the 300 MHz Bruker Avance spectrometer. Chemical shifts referenced to residual solvent signals from [D₆]benzene (7.16 ppm). Fluorine NMR referenced externally with trifluoroacetic acid (−76.8 ppm).

Thermogravimetric Analysis: The TA Q 500 Instrument was used to perform the analyses. Sample sizes between 15 to 30 mg were loaded onto platinum pans. A flow rate of 40 mL/min of purified nitrogen gas passed over the surface of the pan. The temperature was ramped at 10° C. per minute until a final temperature of 700° C.

X-ray data and setup: Single crystal experiments carried out using the Bruker AXS SMART CCD system with three-circle goniometer, graphite-monochromated Mo Kα radiation (λ=0.71073 Å), and narrow frame exposures of 0.3° in θ. A hemisphere of data was collected at low temperatures; cell parameters refined using SMART and integrated using SAINT. The final structures solved and refined using SHELXS-97 and SHELXL-97.

### Example 1

### Synthesis Scheme 1: Alkaline Earth Fluoroalkoxide Monometallics

Identical procedures were used to prepare these compounds. Reactions occurred at room temperature. H(PFTB) (4 mmol, 0.56 mL) was slowly added by syringe into a 100 mL Schlenk tube charged with Ae(HMDS)₂(THF)₂ (Ae=2 mmol, 1.01 g: Ca; 2 mmol, 1.10 g: Sr; 2 mmol, 1.20 g: Ba), in 5 mL of THF. The solution stirred for one hour becoming a pale yellow color for all compounds. After which, diglyme (4 mmol, 0.57 mL) was added with further stirring for an additional hour. The solutions remained pale yellow for all compounds. Compound 3 crystallized as colorless plates from THF in a 5° C. freezer after two days. For compounds 1 and 2, all volatiles were removed under reduced pressure leaving cream colored, powdery residues. A minimal amount of THF (1 mL) re-dissolved the solid and the solution was layered with 10 mL of hexane, then crystallized in a 5° C. freezer. Compound 1 and 2 formed crystals after 24 h.

### Example 2

### Synthesis Scheme 2: Alkaline Earth Fluoroalkoxide Monometallics

A 100 mL Schlenk tube was charged with alkaline earth hexamethyldisilazide (5 mmol), and ammonium PFTB (10 mmol). Sufficient THF was added to dissolve the solids followed by stirring for 2 hours. Reaction completion was followed by FT-IR spectroscopy; if reaction was incomplete additional reaction time was provided. Upon reaction completion, diglyme was added to the solution followed by an additional two hours of stirring. All volatiles were removed under reduced pressure yielding a solid residue. The product residue was recrystallized from a hexane layered THF solution at room temperature.

### Example 3

### Synthesis Scheme 3: Alkaline Earth Fluoroalkoxide Monometallics

The anhydrous ammonia, as prepared above, was recondensed into a 2-neck 500 mL round bottom flask fitted with a dry ice condenser. The flask was charged with alkaline earth metal (5 mmol) and H(PFTB) (10 mmol) in THF cooled to dry ice-acetone bath temperatures (˜⁻78° C.). When ammonia condensed into the flask the solution turned a blue color. The dry ice-acetone bath was removed and the solution was allowed to reflux until the blue solution turned colorless, at which time the ammonia was allowed to evaporate. The solvent was removed under reduced pressure leaving a powdery residue which was recyrstallized from a hexane layered THF solution at room temperature.

### Example 4

### Synthesis Scheme 4: Alkaline Earth Fluoroalkoxide Monometallics (Scheme 4.)

The anhydrous ammonia, as prepared above, was recondensed into a 2-neck 500 mL round bottom flask fitted with a dry ice condenser. The flask was charged with alkaline earth metal (5 mmol) and (NH₄)(PFTB) (10 mmol) in THF cooled to dry ice-acetone bath temperatures (˜⁻78° C.). When ammonia condensed into the flask the solution turned a blue color. The dry ice-acetone bath was removed and the solution was allowed to reflux until the blue solution turned colorless, at which time the ammonia was allowed to evaporate. The solvent was removed under reduced pressure leaving a powdery residue which was recyrstallized from a hexane layered THF solution at room temperature.

### Example 5

### Synthesis Scheme 5: Rare Earth Fluoroalkoxide Monometallics

A 100 mL Schlenk tube was charged with rare earth hexamethyldisilazide (5 mmol), and H (PFTB) (10 mmol). Sufficient THF was added to dissolve the solids followed by stirring for 2 hours. Reaction completion was followed by FT-IR spectroscopy; if reaction was incomplete additional reaction time was provided. Upon completion of the reaction, diglyme was added to the solution followed by an additional two hours of stirring. All volatiles were removed under reduced pressure yielding a solid residue. The product residue was recrystallized from a hexane layered THF solution at room temperature.

### Example 6

### Synthesis Scheme 6: Rare Earth Fluoroalkoxide Monometallics

A 100 mL Schlenk tube was charged with rare earth hexamethyldisilazide (5 mmol), and ammonium (PFTB) (10 mmol). Sufficient THF was added to dissolve the solids followed by stirring for 2 hours. Reaction completion was followed by FT-IR spectroscopy; if reaction was incomplete additional reaction time was provided. Upon reaction completion, diglyme was added to the solution followed by an additional two hours of stirring. All volatiles were removed under reduced pressure yielding a solid residue. The product residue was recrystallized from a hexane layered THF solution at room temperature.

### Example 7

### Synthesis Scheme 7: Rare Earth Fluoroalkoxide Monometallics

The anhydrous ammonia, as prepared above, was recondensed into a 2-neck 500 mL round bottom flask fitted with a dry ice condenser. The flask was charged with rare earth metal (5 mmol) and H(PFTB) (10 mmol) in THF cooled to dry ice-acetone bath temperatures (˜⁻78° C.). When ammonia condensed into the flask the solution turned a blue color. The dry ice-acetone bath was removed and the solution was allowed to reflux until the blue solution turned colorless, at which time the ammonia was allowed to evaporate. The solvent was removed under reduced pressure leaving a powdery residue which was recyrstallized from a hexane layered THF solution at room temperature.

### Example 8

### Synthesis Scheme 8: Rare Earth Fluoroalkoxide Monometallics

The anhydrous ammonia, as prepared above, was recondensed into a 2-neck 500 mL round bottom flask fitted with a dry ice condenser. The flask was charged with rare earth metal (5 mmol) and ammonium (PFTB) (10 mmol) in THF cooled to dry ice-acetone bath temperatures (˜⁻78° C.). When ammonia condensed into the flask the solution turned a blue color. The dry ice-acetone bath was removed and the solution was allowed to reflux until the blue solution turned colorless, at which time the ammonia was allowed to evaporate. The solvent was removed under reduced pressure leaving a powdery residue which was recyrstallized from a hexane layered THF solution at room temperature.

### Example 9

### Synthesis Scheme 9: Alkali/Alkaline Earth Fluoroalkoxide Heterobimetallics

The combination of two separately prepared yielded the desired products. Reactions took place at room temperature. Solution A: H(PFTB) (2 mmol, 0.28 mL) was added by syringe into a 100 mL Schlenk tube containing a cloudy mixture alkali hydride (Na: 2 mmol, 0.05 g; K: 2 mmol, 0.08 g) suspended in 3 mL of THF. Immediate evolution of hydrogen gas was observed, and the solution turned clear within five minutes. However, stirring continued for 1 h. Solution B: PFTB (4 mmol, 0.56 mL) was added by syringe directly into a solution of alkaline earth HMDS (Sr: 2 mmol, 1.11 g; Ba: 2 mmol, 1.20 g) in 2 mL of THF. The only visible change observed was that the pale yellow solution becomes a lighter pale yellow after an hour of stirring. Solutions A and B were then combined and stirred for an additional hour, after which all volatiles were removed under reduced pressure. The white powdery residue was re-dissolved in a minimal amount of THF (1.2 mL) and was then layered with hexane (10 mL). The resulting pale yellow solution crystallized in a 5° C. freezer within a day.

### Example 10

### Synthesis Scheme 10: Alkali/Alkaline Earth Fluoroalkoxide Heterobimetallics

The combination of two separately prepared yielded the desired products. Reactions took place at room temperature. Solution A: (NH₄)(PFTB) (2 mmol) and alkali hydride (2 mmol) were allowed to react in a 100 mL Schlenk tube containing 3 mL of THF. Immediate evolution of hydrogen gas was observed, and the solution turned clear within five minutes. However, stirring continued for 1 h. Solution B: an alkaline earth PFTB complex (2 mmol) was dissolved in 3 mL of THF. Solutions A and B were then combined and stirred for an additional hour, after which all volatiles were removed under reduced pressure. The white powdery residue was re-dissolved in a minimal amount of THF and was then layered with hexane (10 mL). The resulting pale yellow solution crystallized in a 5° C.

### Example 11

### Synthesis Scheme 11: Alkali/Rare Earth Fluoroalkoxide Heterobimetallics

The combination of two separately prepared yielded the desired products. Reactions took place at room temperature. Solution A: H(PFTB) (2 mmol) was added by syringe into a 100 mL Schlenk tube containing a cloudy mixture alkali hydride (2 mmol) suspended in 3 mL of THF. Immediate evolution of hydrogen gas was observed, and the solution turned clear within five minutes. However, stirring continued for 1 h. Solution B: a previously prepared rare earth PFTB complex (2 mmol) was dissolved in 2 mL of THF. Solutions A and B were then combined and stirred for an additional hour, after which all volatiles were removed under reduced pressure. The white powdery residue was re-dissolved in a minimal amount of THF and was then layered with hexane (10 mL). The resulting solution crystallized in a 5° C. freezer.

### Example 12

### Synthesis Scheme 12: Alkali/Rare Earth Fluoroalkoxide Heterobimetallics

The combination of two separately prepared yielded the desired products. Reactions took place at room temperature. Solution A: (NH₄)(PFTB) (2 mmol) and alkali hydride (2 mmol) were allowed to react in a 100 mL Schlenk tube containing 3 mL of THF. Immediate evolution of hydrogen gas was observed, and the solution turned clear within five minutes. However, stirring continued for 1 h. Solution B: a previously prepared rare earth PFTB complex (2 mmol) was dissolved in 3 mL of THF. Solutions A and B were then combined and stirred for an additional hour, after which all volatiles were removed under reduced pressure. The white powdery residue was re-dissolved in a minimal amount of THF and was then layered with hexane (10 mL). The resulting solution crystallized in a 5° C.

### Example 13

### Synthesis Scheme 13: Alkaline Earth/Alkaline Earth Fluoroalkoxide Heterobimetallics

Reactions took place at room temperature. A 100 mL Schlenk tube containing a mixture of two different alkaline earth PFTB complexes (5 mmol of each) was dissolved in THF. The resulting solution was stirred for 5 hours, after which all volatiles were removed under reduced pressure leaving a solid residue. A minimal amount of THF was added to the powder to re-dissolve it and the solution was layered with hexane (10 mL). Crystals were grown at room temperature.

### Example 14

### Synthesis Scheme 14: Alkaline/are Earth Fluoroalkoxide Heterobimetallics

Reactions took place at room temperature. A 100 mL Schlenk tube containing a mixture of an alkaline earth PFTB complex (5 mmol) and a rare earth PFTB complex (5 mmol) were dissolved in THF. The resulting solution was stirred for 5 hours, after which all volatiles were removed under reduced pressure leaving a solid residue. A minimal amount of THF was added to the powder to re-dissolve it and the solution was layered with hexane (10 mL). Crystals were grown at room temperature.

### Example 15

### Synthesis Scheme 15: Rare Earth/Rare Earth Fluoroalkoxide Heterobimetallics

Reactions took place at room temperature. A 100 mL Schlenk tube containing a mixture of two different rare earth PFTB complexes (5 mmol of each) was dissolved in THF. The resulting solution was stirred for 5 hours, after which all volatiles were removed under reduced pressure leaving a solid residue. A minimal amount of THF was added to the powder to re-dissolve it and the solution was layered with hexane (10 mL). Crystals were grown at room temperature.

### Example 16

### Characterization of the Metal Fluoroalkoxide Compounds

Ca(PFTB)₂ (diglyme)₂ (COMPOUND 1): colorless needles; 0.88 g (55.2%) yield; mp=78-9° C.; ¹H NMR(C₆D₆, 300 MHz, ppm) δ=3.07 (s, 6H, —CH₃), 3.14 (m, 4H, —OCH₂—), 3.34 (m, 4H, —OCH₂—); ¹³C NMR(C₆D₆, 300 MHz, ppm) δ=59.2 (s, —OCH₃), 68.7 (s, —OCH₂), 71.2 (s, —OCH₂), none observed for PFTB; ¹⁹F NMR(C₆D₆, 300 MHz, ppm) δ=−75.6; FT-IR (mull, cm⁻¹): 1300 (m), 1258 (m), 1230 (m), 1200 (m), 1140 (w), 1096 (w), 1067 (w), 955 (m), 868 (w), 723 (s), 532 (w).

Sr(PFTB)₂ (diglyme)₂ (COMPOUND 2): colorless blocks; 1.23 g (72.5%) yield; mp=152-3° C.; ¹H NMR(C₆D₆, 300 MHz, ppm) δ=3.08 (m, 4H, —OCH₃), 3.12 (s, 6H, —CH₃), 3.26 (m, 4H, —OCH₂—); ¹³C NMR(C₆D₆, 300 MHz, ppm) δ=59.1 (s, —CH₃), 68.9 (s, —OCH₂), 71.2 (s, —OCH₂), none observed for PFTB; ¹⁹F NMR(C₆D₆, 300 MHz, ppm) δ=−75.9 (s, —CF₃); FT-IR (mull, cm⁻¹): 1254 (m), 1208 (w), 1136 (m), 1103 (w), 1077 (w), 953 (m), 802 (m), 722 (s).

Ba(PFTB)₂ (diglyme)₂ (COMPOUND 3): colorless plates, 1.15 g (65.7%) yield; mp=210° C.; ¹³C NMR(C₆D₆, 300 MHz, ppm) δ=58.8 (s, —CH₃), 69.0 (s, —OCH₂—), 71.6 (s, —OCH₂—), none observed for PFTB; ¹⁹F NMR (C₆D₆, 300 MHz, ppm) δ=−76.0 (s, —CF₃); FT-IR (mull, cm⁻¹): 1304 (m), 1261 (m), 1177 (m), 1021 (w), 955 (s), 724 (s).

Na(THF)Ba(μ-PFTB)₃(THF)₃ (COMPOUND 11): colorless block; 1.24 g (53.5%) yield; mp=210° C.; FT-IR (mull, cm⁻¹): 1300 (m), 1269 (m), 1230 (m), 1178 (w), 1052 (w), 966 (s), 723 (s).

K(THF)Sr(μ-PFTB)₃(THF)₃ (COMPOUND 14): colorless blocks; 1.52 g (74%) yield; mp=94° C.; FT-IR (mul, cm⁻¹): 1300 (m), 1263 (m), 1240 (m), 1204 (m), 1152 (m), 1040 (w), 960 (s), 880 (w), 723 (s), 532 (w); ¹H NMR(C₆D₆, 300 MHz, ppm) δ=1.4 (m, 4H, —CH₂—), 3.55 (m, 4H, —OCH₂—); ¹⁹F NMR(C₆D₆, 300 MHz, ppm) δ=−75.35, −76.39.

K(THF)Ba(μ-PFTB)₃(THF)₃ (COMPOUND 15): colorless blocks; 0.70 g (29.9%) yield; mp=210° C.; FT-IR (mull, cm⁻¹): 1300 (m), 1263 (m), 1230 (m), 1185 (m), 1040 (w), 961 (m), 879 (w), 723 (s), 533 (w).

### PARTS LIST FOR FIGS. 1-8

601 structure of Ca(PFTB)₂(diglyme)₂ (compound 1)
610 structure of Sr(PFTB)₂(diglyme)₂ (compound 2)
620 structure of Ba(PFTB)₂(diglyme)₂ (compound 3)
625 temperature (degrees Celsius)
630 weight (%)
701 compound 11
710 compound 14
720 compound 15
725 temperature (degrees Celsius)
730 weight (%)

While this invention has been particularly shown and described with references to preferred embodiments thereof, it will be understood by those skilled in the art that various changes in form and details may be made therein without departing from the intended scope of the invention encompassed by the following appended claims.

1. A composition of matter comprising a complex between one or more fluoroalkoxide ligands and one or more metals, said complex represented by the formula: 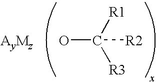
wherein R1, R2 and R3 are fluoroalkyl groups, x, y and z are non-negative integers, wherein y and z are not simultaneously zero, and A and M are metals each selected from the group consisting of alkali metals, alkaline earth metals, lanthanoids and Y.

2. The composition of matter of claim 1, wherein said complex is a heterobimetallic complex.

3. The composition of matter of claim 1, wherein at least two of said fluoroalkyl groups R1, R2 and R3 have a different chemical structure from each other.

4. The composition of matter of claim 1, wherein all of said fluoroalkyl groups R1, R2 and R3 have the same chemical structure.

5. The composition of matter of claim 1, wherein at least one of said fluoroalkyl groups R1, R2 and R3 comprises a fluorinated methyl group.

6. The composition of matter of claim 1, wherein at least one of said fluoroalkyl groups R1, R2 and R3 is fully fluorinated.

7. The composition of matter of claim 1, wherein said fluoroalkoxide ligand is perfluoro-tert-butoxide.

8. The composition of matter of claim 1, wherein said metal A is different from said metal M.

9. The composition of matter of claim 1, wherein said metal A and said metal M belong to the same group.

10. The composition of matter of claim 1, wherein said metal A and said metal M belong to different groups.

11. The composition of matter of claim 1, wherein said complex further comprises a plurality of donor molecules.

12. The composition of matter of claim 11, wherein said donor molecules are selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

13. The composition of matter of claim 7, wherein said complex further comprises a plurality of donor molecules.

14. The composition of matter of claim 13, wherein said donor molecules are selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

15. The composition of matter of claim 1, wherein said complex is non-pyrophoric.

16. The composition of matter of claim 11, wherein said complex is non-pyrophoric.

17. The composition of matter of claim 13, wherein said complex is non-pyrophoric.

18. The composition of matter of claim 1, wherein said complex has an onset of sublimation at a temperature of at most 240° C. at atmospheric pressure.

19. The composition of matter of claim 11, wherein said complex has an onset of sublimation at a temperature of at most 240° C. at atmospheric pressure.

20. The composition of matter of claim 13, wherein said complex has an onset of sublimation at a temperature of at most 240° C. at atmospheric pressure.

21. The composition of matter of claim 13, wherein said metals A and M are each selected from the group consisting of Be, Mg, Ca, Sr and Ba, and x is at least equal to 4, y is equal to 1 and z is equal to 1.

22. The composition of matter of claim 13, wherein said metals A and M are each selected from the group consisting of La, Ce, Pr, Nd, Pm, Eu, Sm, Gd, Tb, Dy, Ho, Er, Tm, Yb, Lu and Y and x is at least equal to 4, 5 or 6, y is equal to 1 and z is equal to 1.

23. The composition of matter of claim 13, wherein said metal A is selected from the group consisting of Be, Mg, Ca, Sr, and Ba and said metal M is selected from the group consisting of Li, Na, K, Rb and Cs, and x is at least equal to 3, y is equal to 1 and z is equal to 1.

24. The composition of matter of claim 13, wherein said metal A is selected from the group consisting of La, Ce, Pr, Nd, Pm, Eu, Sm, Gd, Tb, Dy, Ho, Er, Tm, Yb, Lu and Y and M is a metal selected from the group consisting of Li, Na, K, Rb, and Cs, and x is at least equal to 3 or 4, y is equal to 1 and z is equal to 1.

25. The composition of matter of claim 13, wherein said metal A is selected from the group consisting of La, Ce, Pr, Nd, Pm, Eu, Sm, Gd, Tb, Dy, Ho, Er, Tm, Yb, Lu and Y and M is a metal selected from the group consisting of Be, Mg, Ca, Sr and Ba, and x is at least equal to 4 or 5, y is equal to 1 and z is equal to 1.

26. The composition of matter of claim 13, wherein said metal A is selected from the group consisting of La, Ce, Pr, Nd, Pm, Eu, Sm, Gd, Tb, Dy, Ho, Er, Tm, Yb, Lu and Y, y is at least equal to 1, z is equal to zero, and x is at least equal to 2 or 3.

27. The composition of matter of claim 13, wherein said metal A is selected from the group consisting of Be, Mg, Ca, Sr and Ba, y is at least equal to 1, z is equal to zero, and x is at least equal to 2.

28. A method for chemical vapor deposition on a substrate comprising the steps of:
a) preparing a precursor solution comprising a composition of matter of claim 11;
b) placing said precursor solution in a reactor that is in communication with a substrate;
c) vaporizing said precursor solution to form molecular species in the vapor state; and
d) decomposing said molecular species in the vapor state to deposit a metallic constituent thereof on said substrate.
wherein said decomposition of said molecular species in the vapor state on said substrate results in the deposition of said one or more metals within said composition of matter on said substrate.

29. The method of claim 28, wherein said precursor solution further comprises one or more donor molecules.

30. The method of claim 29, wherein said donor molecules are selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

31. The method of claim 28, wherein said vaporizing of said precursor solution occurs at atmospheric pressure.

32. The method of claim 28, wherein said vaporizing of said precursor solution occurs without oligomerization.

33. The method of claim 28, wherein said decomposition of said molecular species in the vapor state on said substrate results from decomposition of said precursor in contact with said substrate.

34. The method of claim 28, wherein said precursor solution has an onset of sublimation of at a temperature of at most 240° C. at atmospheric pressure.

35. The method of claim 28, wherein said substrate comprises a crystalline material.

36. The method of claim 35, wherein said crystalline material is a silicon crystalline material.

37. The method of claim 35, wherein said precursor solution is non-pyrophoric.

38. A method for chemical vapor deposition on a substrate comprising the steps of:
a) preparing a precursor solution comprising a composition of matter of claim 13;
b) placing said precursor solution in a reactor that is in communication with a substrate;
c) vaporizing said precursor solution to form molecular species in the vapor state; and
d) decomposing said molecular species in the vapor state to deposit a metallic constituent thereof on said substrate,
wherein said decomposition of said molecular species in the vapor state on said substrate results in the deposition of said one or more metals within said composition of matter on said substrate.

39. The method of claim 38, wherein said precursor solution further comprises one or more donor molecules.

40. The method of claim 39, wherein said donor molecules are selected from the group consisting of tetraglyme donors, triglyme donors, diglyme donors, dimethoxyethane (DME) donors, tetrahydrofuran (THF) donors, N,N,N,′N′,′N″-pentamethyltriethylenediamine (PMTDA) donors, and N,N,N,′N′-tetramethlyethylenediamine (TMEDA) donors.

41. The method of claim 38, wherein said vaporizing of said precursor solution occurs at atmospheric pressure.

42. The method of claim 38, wherein said vaporizing of said precursor solution occurs without oligomerization.

43. The method of claim 38, wherein said decomposition of said molecular species in the vapor state on said substrate results from decomposition of said precursor in contact with said substrate.

44. The method of claim 38, wherein said precursor solution has an onset of sublimation of at a temperature of at most 240° C. at atmospheric pressure.

45. The method of claim 38, wherein said substrate comprises a crystalline material.

46. The method of claim 45, wherein said crystalline material is a silicon crystalline material.

47. The method of claim 38, wherein said precursor solution is non-pyrophoric.
